(19) United States
(12) Patent Application Publication
(10) Pub. No.: US 2021/0260274 A1
(43) Pub. Date: Aug. 26, 2021

(54) DEVICE FOR IRRIGATING BODY CAVITIES, FOR EXAMPLE FOR HYSTEROSCOPY

(71) Applicant: W.O.M. WORLD OF MEDICINE GMBH, Berlin (DE)

(72) Inventor: Henrik STIER, Berlin (DE)

(21) Appl. No.: 17/252,352

(22) PCT Filed: Jun. 17, 2019
(86) PCT No.: PCT/IB2019/055042

§ 371 Date: Dec. 15, 2020

(30) Foreign Application Priority Data
Jun. 15, 2018 (DE) 10 2018 004 758.0

(51) Int. Cl. A61M 3/02

(52) U.S. Cl. CPC A61M 3/022; A61M 3/0287; A61M 2205/36; A61M 2205/18; A61M 2205/3334; A61M 2210/1433

(57) ABSTRACT

The present patent application relates to a device for rinsing body cavities, as they are used, for instance, in the hysteroscopy, that determines, by using flow sensors, the introduced and discharged amount of rinsing liquid. Based on these flow measurements, it can be determined whether a relevant amount of liquid has been left in the patient.

### BRIEF DESCRIPTION OF THE DRAWINGS

The present patent application relates to a device for rinsing body cavities, as they are used, for instance, in the hysteroscopy, that determines, by using flow sensors, the introduced and discharged amount of rinsing liquid. Based on these flow measurements, it can be determined whether a relevant amount of liquid has been left in the patient.

Hysteroscopy, named as an example, is a transvaginal inspection of the uterine cavity and the tubal ostia by means of an endoscope. In gynecology, the hysteroscopy is used for diagnostics, but also during surgical interventions. By means of the hysteroscopy, for instance, reasons for bleeding disturbances, myomas or polyps can be found, but also carcinomas (e.g., endometrial carcinoma or cervix carcinoma) can be diagnosed. Myomas and polyps can, for instance, also be removed. Further, endometrium can also be removed for the purpose of examination. For potential hemostasis, high-frequency current can be used.

During these hysteroscopic examinations or surgeries, the uterus is expanded and rinsed using a rinsing solution. Such rinsing solutions may be comprised of, for instance, sodium chloride (saline solution) or sorbitol/manitol (sugar solution).

It is important, for hysteroscopic examinations and interventions, to remove the used rinsing solution as completely as possible from the body. Rinsing solutions remaining in the body may cause a toxic syndrome (TUR syndrome).

According to the hysteroscopic rinsing devices of the prior art, the difference between introduced rinsing solution and discharged rinsing solution is determined by weighing. For instance, the storage bag with the rinsing solution is weighed before, during and after the treatment. The discharged rinsing solution is received in a collection container, which is also weighed before, during and after the surgery. From the difference between introduced and discharged solution, the amount of rinsing solution left in the patient can be concluded.

This method of determination includes a series of disadvantages. In the practice, the operators will make weighing faults, in particular when several bags with rinsing liquid are used or when the collection container must be replaced during the treatment. Further errors may be caused by depositing objects on the weighing scale, by leaning against or moving the weighing system during the surgery. Furthermore, the necessary devices and weighing systems cannot be integrated in the other hospital equipment. For this purpose, separate holding and deposition racks are needed. The relevant state of the art results from the documents DE 4417189 A1, EP 0755282 B1, and EP 1175238 B1. DE 4417189 A1 describes a comparable system for the hysteroscopy with a mechanical flow measurement device. The mechanical flow measurement device is based on that the inflowing or outflowing liquid is conducted through a glass body with a conical inner borehole. A floating body within the cone indicates the flow rate, from which the total flow is calculated. The accuracy of the measurement leaves much to be desired, however, in particular when the liquid comprises blood, air bubbles and/or tissue remainders.

### SUMMARY OF THE INVENTION

It is therefore the object of the present invention to avoid the disadvantages of the above prior art and to provide a device that is easier to use, allows an integration in the usual hospital equipment and at the same time achieves at least equal, if not better results.

It has been found that, surprisingly, the use of ultrasonic flow sensors permits a precise measurement of the introduced and discharged amount of rinsing liquid in the hysteroscopy. As described below, the measurement accuracy can be increased by using such ultrasonic flow sensors, compared to the conventional weighing technique. This was very surprising, since it was assumed that the flow sensors would sensitively respond to changes in the solution, in particular to the admixture of tissue remainders, blood, air bubbles, etc. It turned out, however, that the flow sensors are sufficiently accurate, even under practical conditions, in order to be able to perform the difference determination with sufficient precision.

### BRIEF DESCRIPTION OF THE DRAWINGS

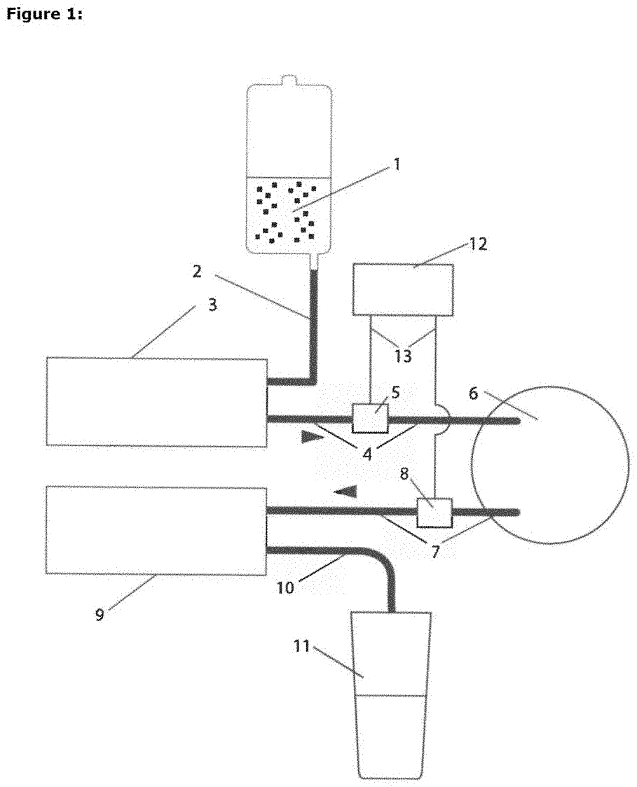

FIG. 1 illustrates a first embodiment of the invention with a storage container with rinsing liquid (1), a first tube connection (2), a first medical pump (3), a second tube connection (4), a first flow sensor (5), the body cavity (6), a third tube connection (7), a second flow sensor (8), a second medical pump (9), a fourth tube connection (10), a collection container (11), an evaluation unit (12), cable connections from the flow sensors to the evaluation unit (13) and a vacuum pump (14). The arrows indicate the liquid flow direction.

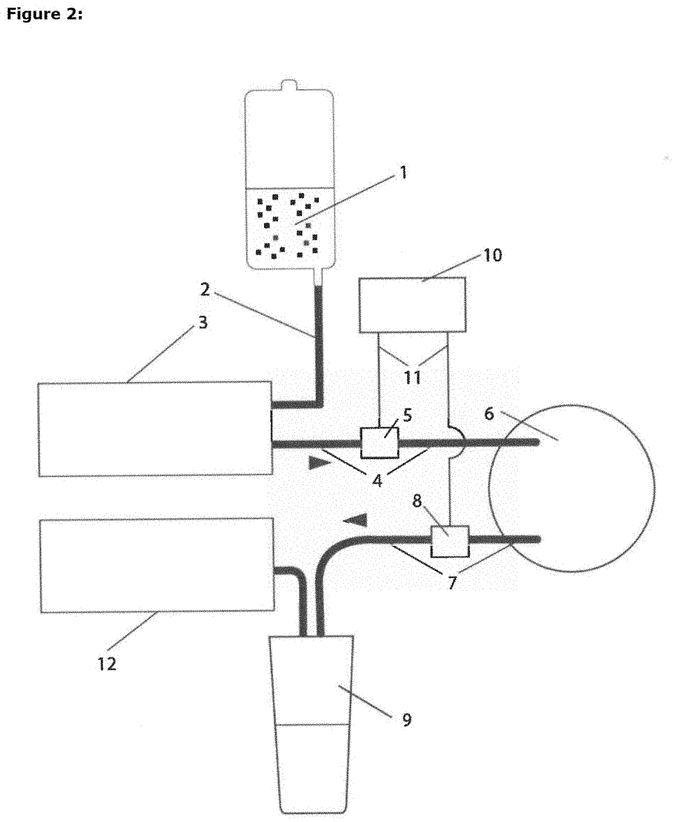

FIG. 2 illustrates a second embodiment of the invention. In contrast to the embodiment of FIG. 1 the a vacuum pump (14) is connected to the collection container (11). In this embodiment the use use of an external vacuum pump is possible. Also in FIG. 2 the arrows indicate the liquid flow direction.

### DETAILED DESCRIPTION OF THE INVENTION

The present invention relates, therefore, to a medical device for rinsing body cavities,

comprising

at least one storage container with rinsing liquid,

a first tube connection to a medical pump,

a second tube connection from the medical pump to the body cavity,

a third tube connection from the body cavity to a second medical pump,

a fourth tube connection from the second medical pump to a collection container,

characterized by

that a first ultrasonic flow sensor is fitted from outside onto the first or the second tube connection,

that a second ultrasonic flow sensor is fitted from outside onto the third or the fourth tube connection, and

that the ultrasonic flow sensors are connected to an evaluation unit that measures the flow rates of both ultrasonic flow sensors and indicates the difference of the flow rates.

The device according to the invention is built up, in principle, as follows:

A storage container contains the required rinsing liquid. From the storage container, the liquid is guided through a first tube connection to a medical pump, e.g., a roller pump. The outlet of the medical pump leads via a second tube connection to the body cavity (e.g., uterus of the patient).

One of the tube connections is provided with an ultrasonic flow sensor. In the preferred case of use of a roller pump, the first and the second tube connections may be formed by a single tube. The ultrasonic flow sensor may be arranged in the direction of flow before or behind the roller pump.

A third tube connection leads from the body cavity (e.g., uterus) outwardly to a second medical pump, e.g., another roller pump. Alternatively, a vacuum pump may be connected to the collection container, as shown in FIG. 2. The vacuum pump may be integrated in a roller pump or may be an external pump. In any case, the second medical pump extracts liquid from the uterus. The outlet of the second medical pump leads into a collection container. The third or the fourth tube connection is also provided with an ultrasonic flow sensor. In the preferred case of use of a roller pump, the third and the fourth tube connection may be formed by a single tube. Here, too, the ultrasonic flow sensor may be arranged in the direction of flow before or behind the roller pump.

The ultrasonic flow sensors are connected by wires or without wires (e.g., by Bluetooth® or wi-fi) to an evaluation unit. This evaluation unit determines the amount of introduced rinsing solution and the amount of discharged rinsing solution and indicates the difference of the amounts. Optionally, of course, the absolute amounts of the introduced rinsing liquid and of the discharged rinsing liquid may also be indicated. If the determined difference is greater than a threshold value adjusted or configured at the system before, in a preferred embodiment of the invention, an alarm signal is initiated. The threshold value may, for instance, be 2500 ml for a saline solution or 1000 ml for sorbitol/mannitol-containing solutions. In a particularly preferred embodiment of the invention, ultrasonic flow sensors are used, which are simply fitted from outside at an arbitrary location onto the mentioned tubes. The sensors may be used several times, e.g., for several examinations per day without having to be sterilized in the meantime. There is, in this case, no risk of any contaminations. The tubes are made of conventional tube materials, as they are used in the medical technology, in particular hysteroscopy. The used tubes are usually designed for single use and are disposed of after use, so that there is no risk of contamination.

It is understood, of course, that the two roller/vacuum pumps and the evaluation unit may be integrated in a single housing.

Optionally, for liquid supply, a pre-heating of the rinsing liquid may also be provided, for instance, by a corresponding flow heater. An example for such a flow-heating device is described in WO 2018/019317 A1.

Ultrasonic flow sensors that are suitable for the purpose according to the invention are known from other technical fields and are commercially available (e.g., Sonoflow Clamp-on Sensor of Sonotec GmbH, SonoTT Clamp-on Transducer of Em-tec GmbH, BioPro of Em-tech GmbH, or Sonoc of CeramTec GmbH) and do not need any further explanation here.

In experiments, it has been found that the accuracy of the measurement is significantly better than when using the systems described above, also and especially when using several bags with rinsing liquid. In hysteroscopic surgeries, it is not uncommon that the total amount of rinsing liquid during a surgery is 20 l or more (i.e., e.g., 4 bags of 5000 ml each).

The device according to the invention may be used in different clinical applications: In addition to the hysteroscopy named as an example, in particular also urological applications (e.g., transurethral resection of the bladder or prostate (TUR) or for the urethral enucleation) come into consideration.

The invention is illustrated exemplarily by the scheme in FIG. 1.

The reference numerals used in FIG. 1 and FIG. 2 have the following meanings:

1 storage container with rinsing liquid

2 first tube connection

3 first medical pump

4 second tube connection

5 first flow sensor

6 body cavity

7 third tube connection

8 second flow sensor

9 second medical pump

10 fourth tube connection

11 collection container

12 evaluation unit

13 cable connections from the flow sensors to the evaluation unit

14 vacuum pump

The two black triangles at the tube connections 4 or 7 indicate the direction of flow of the rinsing liquid.

1. A medical device for rinsing body cavities, comprising
at least one storage container with rinsing liquid,
a first tube connection to a medical pump,
a second tube connection from the medical pump to the body cavity,
a third tube connection from the body cavity to a second medical pump,
a fourth tube connection from the second medical pump to a collection container,
characterized by
that a first ultrasonic flow sensor is fitted from outside onto the first or the second tube connection,
that a second ultrasonic flow sensor is fitted from outside onto the third or the fourth tube connection, and
that the ultrasonic flow sensors are connected to an evaluation unit that measures the flow rates of both ultrasonic flow sensors and indicates the difference of the flow rates.

2. The medical device according to claim 1, wherein the medical pumps are roller pumps.

3. The medical device according to claim 1, wherein the evaluation unit initiates an alarm signal, when the measured difference of the flow rates exceeds a pre-determined threshold value.

4. The medical device according to claim 1, wherein the pre-determined threshold value is 500, 750, 1000, 1250, 1500, 1750, 2000, 2250, or 2500 ml.

5. The medical device according to claim 1, wherein in the liquid supply, a temperature control device is provided.

6. A method of performing a hysteroscopy or urology procedure using a medical device according to claim 1.
